(19) United States
(12) Patent Application Publication
(10) Pub. No.: US 2023/0416679 A1
(43) Pub. Date: Dec. 28, 2023

(54) METHODS AND COMPOSITIONS FOR IN-VITRO AUGMENTATION OF MILK PRODUCTION FROM MAMMARY EPITHELIAL CELLS

(71) Applicants: Wilk Technologies International Ltd., Rehovot (IL); Yissum Research Development Company of the Hebrew University of Jerusalem Ltd., Jerusalem (IL)

(72) Inventor: Nurit ARGOV-ARGAMAN, Tel-Aviv (IL)

(73) Assignees: Wilk Technologies International Ltd., Rehovot (IL); Yissum Research Development Company of the Hebrew University of Jerusalem Ltd., Jerusalem (IL)

(21) Appl. No.: 18/368,070

(22) Filed: Sep. 14, 2023

Related U.S. Application Data
(63) Continuation of application No. PCT/IL2022/050307, filed on Mar. 17, 2022.
(60) Provisional application No. 63/162,040, filed on Mar. 17, 2021.
(60) Provisional application No. 63/302,633, filed on Jan. 25, 2022.

(51) Int. Cl. C12N 5/071; A23L 33/125; A23L 33/19

(52) U.S. Cl. CPC C12N 5/0631; A23L 33/125; A23L 33/19; C12N 2500/36; C12N 2501/999; C12N 2500/32

(57) ABSTRACT

A method of increasing milk secretion of a predetermined milk component from a tissue culture comprising mammary epithelial cells (MECs) is provided. The method comprising admixing into a medium composition comprising the tissue culture, a biologically effective concentration of a composition selectably operable to increase the secretion of the predetermined components, the composition comprising at least one agent selected from the group consisting of oleic acid, beta-hydroxybutirate (BHBA) and a phenolic composition, wherein the agent is not an ethanol extract of P. lentiscus; and when the agent comprises oleic acid it comprises at least two of the agents.

### RELATED APPLICATIONS

This application is a Continuation of PCT Patent Application No. PCT/IL2022/050307 having International filing date of Mar. 17, 2022, which claims priority under 35 USC § 119(e) of U.S. Provisional Patent Application No. 63/302,633 filed on Jan. 25, 2022 and U.S. Provisional Patent Application No. 63/162,040 filed on Mar. 17, 2021. The contents of the above applications are all incorporated by reference as if fully set forth herein in their entirety.

### FIELD AND BACKGROUND OF THE INVENTION

The present invention, in some embodiments thereof, relates to methods, systems and compositions for use in the ex-vivo production of milk. More specifically, the disclosure is directed to systems, compositions and methods for in-vitro augmentation of milk fat, protein and carbohydrate production using mammary epithelial cells' cultures.

The global dairy market, comprising the processing and harvesting of animal milk for human consumption, reached a value of US$718.9 Billion in 2019, and is typically sourced from cow, goat, buffalo, camel and sheep. With widespread demand for dairy products and their proactive function in the global food industry, dairy plays a crucial role in the growth of the economies worldwide.

Existing dairy milk alternatives, such as soy, almond, rice, or coconut milk fall short both in flavor and in functionality; moreover, a large part of the industrial and cultural significance of dairy milk stems from its usefulness in derivative products, such as cheese, yogurt, cream, or butter. Non-dairy plant-based milks, while addressing environmental and health concerns (and while providing adequate flavor for a small segment of the population), almost universally fail to form such derivative products when subjected to the same processes used for dairy milk.

Moreover, recent report from IATP noted, that as of 2017, the 13 top dairy companies' emissions grew 11% compared with 2015, corresponding to a 32.3 million metric ton increase in greenhouse gases equivalent to the emissions that would be released by adding an extra 6.9 million cars to the road for a year.

Mammary gland epithelial cells (MECs) can be cultured to synthesize and secret milk components to a given medium. Commonly used commercial growth medium usually include amino acids, essential fatty acids and glucose or pyruvate, which are intended to provide the cells' nutritional needs for production of milk components (and milk). See for example, Nan et al. Physiol Genomics 46: 268-275, 2014. Nevertheless the secretion capacity is rather low, compared with in-vivo quantities, especially that of milk protein, milk fat and lactose.

Additional background art includes:
Cohen et al. 2015 PLoS ONE 10(3): e0121645
Cohen et al. 2017 Journal of Mammary Gland Biology and Neoplasia (2017) 22:235-249
Hadaya, O., et al. “Pistacia lentiscus extract enhances mammary epithelial cells' productivity by modulating their oxidative status.” Scientific reports 10.1 (2020): 1-16).

### SUMMARY OF THE INVENTION

According to an aspect of some embodiments of the present invention there is provided a method of increasing milk secretion of a predetermined milk component from a tissue culture comprising mammary epithelial cells (MECs), the method comprising admixing into a medium composition comprising the tissue culture, a biologically effective concentration of a composition selectably operable to increase the secretion of the predetermined components, the composition comprising at least one agent selected from the group consisting of oleic acid, β-hydroxybutirate (BHBA) and a phenolic composition, wherein the agent is not an ethanol extract of P. lentiscus; and when the agent comprises oleic acid it comprises at least two of the agents.

According to an aspect of some embodiments of the present invention there is provided a composition for selectably increasing milk secretion of predetermined milk component from a tissue culture comprising mammary epithelial cells (MECs), the composition comprising a base medium and a biologically effective concentration of a composition selectably operable to increase the secretion of the predetermined component, the composition comprising at least one agent selected from the group consisting of oleic acid, β-hydroxybutirate (BHBA) and a phenolic composition, wherein the agent is not an ethanol extract of P. lentiscus; and when the agent comprises oleic acid it comprises at least two of the agents.

According to some embodiments of the invention, the method further comprises harvesting secretome of the MECs.

According to some embodiments of the invention, the predetermined components are milk lipids.

According to some embodiments of the invention, the milk lipids comprise triglycerides.

According to some embodiments of the invention, the effective concentration of oleic acid is about 100-1000 μM or 50-360 μM.

According to some embodiments of the invention, when the agent comprises oleic acid it comprises BSA.

According to some embodiments of the invention, a concentration of the BSA is between about 0.5-32 mg BS A/ml medium.

According to some embodiments of the invention, the agent comprises at least 2 agents comprising oleic acid and BHBA

According to some embodiments of the invention, the biologically effective concentration of the BHBA is between about 0.5-2.5 mM.

According to some embodiments of the invention, the phenolic composition is selected from the group consisting of a flavonol, flavanol, flavone, flavanone and an anthocyanidin.

According to some embodiments of the invention, the phenolic composition is synthetic.

According to some embodiments of the invention, the phenolic composition is purified from a natural source.

According to some embodiments of the invention, the phenolic composition is gallic acid or derivative thereof.

According to some embodiments of the invention, the biologically effective concentration of the gallic acid is between about 1 ppm and 3 ppm.

According to some embodiments of the invention, the predetermined components are milk carbohydrates.

According to some embodiments of the invention, the milk carbohydrate is lactose.

According to some embodiments of the invention, the at least one agent is gallic acid or myricetin or derivative thereof.

According to some embodiments of the invention, the predetermined components are milk proteins.

According to some embodiments of the invention, the milk protein is at least one of: whey protein, α-s1, α-s2, β, and 6 casein.

According to some embodiments of the invention, the at least one agent is myricetin or derivative thereof.

According to some embodiments of the invention, the agent further comprises at least one of pyruvate, amino acid and an amino acid dipeptide.

According to some embodiments of the invention, the amino acid (AA) agent comprises Lysine (Lys), Methionine (Met), Threonine (Thr), Phenylalanine (Phe), Leucine (Leu), Isoleucine (Ile), Valine (Val), or Histidine (His) at predetermined ratios.

According to some embodiments of the invention, the effective concentration of the at least one of the amino acids is each between about 45 μg AA/ml medium, and about 215 μg/ml.

According to some embodiments of the invention, the dipeptide is a Met-Met dipeptide.

According to some embodiments of the invention, the predetermined AA ratio is: Lys:Met 2.9:1; Thr:Phe 1.05:1; Lys:Thr 1.8:1; Lys:His 2.38:1; Lys:Val 1.23:1.

According to some embodiments of the invention, the MECs comprise a primary culture of MECs.

According to some embodiments of the invention, the MECs comprise a MEC line.

According to some embodiments of the invention, the MECs comprise an immortalized MEC line.

According to some embodiments of the invention, the MECs comprise a MEC monolayer.

According to some embodiments of the invention, the MECs comprise a MEC organoid.

According to some embodiments of the invention, the MECs comprise a MEC tissue.

According to some embodiments of the invention, the MECs are of a bovine or human source.

According to some embodiments of the invention, the MECs comprise ex vivo differentiated MECs.

According to an aspect of some embodiments of the present invention there is provided a composition comprising a secretome obtainable according to the method of as described herein.

According to an aspect of some embodiments of the present invention there is provided a food or feed comprising the secretome as described herein.

According to an aspect of some embodiments of the present invention there is provided a method of producing food or feed comprising combining the composition as described herein in a food production process.

According to an aspect of some embodiments of the present invention there is provided a method of increasing milk secretion of predetermined milk component from a tissue culture comprising mammary epithelial cells (MECs), the method comprising admixing into a medium composition comprising the tissue culture, a biologically effective concentration of a composition selectably operable to increase the secretion of the predetermined components.

According to some embodiments of the invention, the predetermined components are milk lipids.

According to an aspect of some embodiments of the present invention there is provided a composition for selectably increasing milk secretion of predetermined milk component from a tissue culture comprising mammary epithelial cells (MECs), the composition comprising a biologically effective concentration of a composition selectably operable to increase the secretion of the predetermined component.

According to some embodiments of the invention, the composition selectably operable to increase lipid production from the MECs culture comprises at least one of: an oleic, and a palmitic acid, and bovine serum albumin (BSA).

According to some embodiments of the invention, the composition selectably operable to increase lipid production from the MECs culture further comprises, a biologically effective concentration of β-hydroxybutirate (BHBA).

According to some embodiments of the invention, the composition selectably operable to increase lipid production from the MECs culture further comprises a biologically effective concentration of gallic acid.

According to some embodiments of the invention, the biologically effective concentration of the at least one of: oleic, and palmitic acids is between about 50 μM and about 360 μM, and the BSA concentration is between about 0.5 mg BSA/ml Medium and about 32 mg/ml.

According to some embodiments of the invention, the biologically effective concentration of the BHBA is between about 0.5 mM and about 2.5 mM.

According to some embodiments of the invention, the biologically effective concentration of the gallic acid is between about 1 ppm and 3 ppm.

According to some embodiments of the invention, the method further comprises:
a) analyzing the concentration of trans-10, cis-12 conjugated linoleic acid (CLA) in the secreted milk; and
b) upon determining the concentration of trans-10, cis-12 CLA is over a predetermined threshold, removing any excess trans-10, cis-12 CLA.

According to some embodiments of the invention, the predetermined components are milk proteins.

According to some embodiments of the invention, the milk protein is at least one of: whey protein, α-s1, α-s2, β, and 6 casein.

According to some embodiments of the invention, the medium composition comprising the tissue culture further comprises an effective concentration of an amino acid composition.

According to some embodiments of the invention, the amino acid (AA) composition comprises Lysine (Lys), Methionine (Met), Threonine (Thr), Phenylalanine (Phe), Leucine (Leu), Isoleucine (Ile), Valine (Val), and Histidine (His) at predetermined ratios.

According to some embodiments of the invention, the effective concentration of the at least one of the amino acids is each between about 45 μg AA/ml medium, and about 215 μg/ml.

According to some embodiments of the invention, the composition selectably operable to increase protein production from the MECs culture comprises an effective amount of myrecitin.

According to some embodiments of the invention, the method further comprises replacing between about 5% (w/w) and about 30% of the methionine with a Met-Met dipeptide.

According to some embodiments of the invention, the predetermined AA ratio is: Lys:Met 2.9:1; Thr:Phe 1.05:1; Lys:Thr 1.8:1; Lys:His 2.38:1; Lys:Val 1.23:1.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the Drawings:

FIGS. 1A-B show the effect of oleic acid combined with BSA on lipid production by mammary epithelial cells. FIG. 1A shows that oleic acid increases triglyceride secretion to the medium by primary culture of bovine mammary epithelial cells. Values represent mean±standard error (n=3). Different letters indicate significant difference, P≤0.05. FIG. 1B shows representative images of fat droplets stained with Nile red from conditioned medium collected after 24 h of incubating mammary epithelial cells with oleic acid (right panel) compared with control medium (no oleic acid, left panel).

FIG. 2 demonstrates the effect of different doses of oleic acid on mammary epithelial cells fat production capacity. Fat is stained in red (Nile red) and nucleus in blue (Dapi).

FIG. 3 demonstrates the triglyceride content in MEC exposed for 48 hours to 360 μM oleic acid (O) with or without 1.2 mM betahydroxybutyrate (H);

FIG. 4 demonstrates the elevated content of lipids in MEC treated with 1 or 10 ppm ethyl-acetate extract for 24 h compared with control (not treated cells). Ethyl-acetate extract composed primarily of myrecitin, rutin, ethyl gallate and gallic acid;

FIG. 5 demonstrates the effect of 1 and 10 ppm of ethyl-acetate extract on the levels of lipid inside the cell. The results represent the fold of change compared with control. The lipid content in MEC was determined by the mean diameter of intracellular lipid droplets visualized under fluorescence microscopy (left panel) and the triglyceride concentration as determined by HPLC equipped with ELSD;

FIG. 6 demonstrates the effect of gallic acid on the content of triglyceride in MEC. Triglyceride content is determined by Nile red staining of the intracellular lipid droplets;

FIG. 7A-D demonstrate the effect of 1 ppm gallic acid compared with 10 ppm myrecitin on intracellular triglyceride content (upper panel) or lactose content (lower panel) in MEC after 24 h of treatment. Values represent the fold change in the content of triglyceride and lactose compared with control (untreated MEC).

### DESCRIPTION OF SPECIFIC EMBODIMENTS OF THE INVENTION

The present invention, in some embodiments thereof, relates to methods, systems and compositions for use in the ex-vivo production of milk. More specifically, the disclosure is directed to systems, compositions and methods for in-vitro augmentation of milk fat, protein and carbohydrate production using mammary epithelial cells' cultures.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Mammary gland epithelial cells can be cultured and synthesize to secret milk components to the medium. Commonly used commercial growth medium usually include amino acids, essential fatty acids and glucose or pyruvate, which are intended to provide the cellular nutritional needs for production of milk components. Nevertheless the secretion capacity is rather low, compared with in vivo secretion, especially that of milk protein, fat and lactose. In certain exemplary implementations, provided herein are methods and compositions configured to enhance production of milk components, by formulating a unique media which will include lipogenic building blocks to enhance production of de novo synthesized fatty acids, and of specific polyphenolic compound that was found to increase milk protein synthesis in these cells.

Additional limiting factor for production of milk in MEC is reactive compounds like reactive oxygen species (ROS) which are produced naturally as part of the energy production process in the cells. In mammary gland, the synthesis and secretion of milk constituents occurs in mammary epithelial cells (MEC), a process which requires high metabolic rates and which is manifested by elevated ROS levels. The accumulation of excessive ROS production and accumulation may impair the cellular capacity to produce energy or utilize oxygen for proper protein folding in the endoplasmic reticulum (ER), and reduce production of milk constituents. To alleviate the detrimental effect of ROS on production, specific polyphenol compounds are utilized, which can neutralize ROS and enable MEC to utilize energy and other resources for milk components' production rather than for damage (to the cell) control. Selection of the phenolic composition can govern the secreted milk component.

Thus, according to an aspect of the invention, there is provided a method of increasing milk secretion of a predetermined milk component from a tissue culture comprising mammary epithelial cells (MECs), the method comprising admixing into a medium composition comprising the tissue culture, a biologically effective concentration of a composition selectably operable to increase the secretion of the predetermined components, said composition comprising at least one agent selected from the group consisting of oleic acid, β-hydroxybutirate (BHBA) and a phenolic composition, wherein said agent is not an ethanol extract of P. lentiscus; and when said agent comprises oleic acid it comprises at least two of said agents.

Mammary epithelial cells secrete milk constituents by several routes. Milk lipid is enveloped by a milk fat globule membrane (MFGM) derived from the apical cell surface, and contains some of its constituent proteins. Soluble milk proteins are secreted by exocytosis.

As used herein “milk component” refers to a chemical or a combination of chemicals which are secreted by mammary epithelial cells and are found naturally in milk. For example, proteins (e.g., caseins, e.g., α casein, β casein, and α lactalbumin and whey), lipids (including but not limited to triglycerides), carbohydrates (e.g., lactose or oligosaccharides), Vitamins (including Vitamin A, Vitamin D3, Vitamin E, Vitamin K, Thiamin, Riboflavin, Niacin, Vitamin B6, Vitamin B12. Pantothenic acid, folic acid, Vitamin C and Biotin), minerals (including iron, calcium, phosphorus, magnesium, sodium, chloride, potassium, manganese, iodine, selenium, copper and zinc), choline, myoinositol and L-carnitine.

According to a specific embodiment, the milk component is a lipid, a protein, a carbohydrate or a combination of same, e.g., lipid+carbohydrate, lipid+protein, carbohydrate+protein.

Methods of analyzing a milk component typically include, but are not limited to the use of staining, mass-spectrometry and chromatography.

For instance, in order to analyze lipid secretion, total lipids are extracted from the collected medium. When intracellular lipids are analyzed the same can be done on lysed cells. Though it will be appreciated that the intracellular lipid composition may be different from the secreted lipids. The medium or the cells are diluted in Folch reagent (Chloroform-Methanol 2:1). After overnight incubation with cold extraction at 4° C., the upper phase is removed, and the lower phase is filtered through glass wool. Samples are then evaporated under a nitrogen stream at 65° C., diluted in chloroform:methanol (97:3, v/v) and stored at −20° C. until injection for HPLC analysis. Separation of polar and neutral lipids is performed on a silica column using with an evaporative light-scattering detector. The specific running conditions are described in the literature or in the Examples section which follows. The separated lipids are identified using external standards. Quantification can be performed against external standard curves and expressed as μg/per 10⁶ live cells or as weight % out of the sum of phospholipids (μg) in the sample. Live cell number can be determined with a hemocytometer using trypan blue staining.

HPLC equipped with a UV detector can also be used for detection of protein secretion. Protein content is determined in 220 nm wavelength by C-18 reverse phase column.

HPLC equipped with a refractive index detector at 68° C. is typically used to detect secreted lactose.

As used herein “milk secretion” refers to secretion of the milk component to the medium. In some instances, this may be referred to as a “conditioned medium”. However, in other instances, the nutrient medium on which the cells feed upon (are cultured in) is separated from the medium to which the cells secrete the milk component so as to allow harvesting of the milk secretion (secretome), as further described hereinbelow.

As used herein “increasing” refers to an increase by at least 10%, 20%, 30%, 40%, 50%, 70%, 90% or more, say 1.2 fold, 1.5 fold, 2 fold, 5 fold, 10 fold or more as compared to MECs which are cultured under control conditions i.e., not subjected to the agent, but otherwise same conditions.

As used herein “tissue culture” refers to ex vivo growth of cells.

The cells can be isolated single cells, cell clumps (e.g., organoids) or comprised in a tissue which typically comprises more than one cell type, e.g., mammary epithelial cells and mammary stromal cells (mainly fibroblast and progenitor cells of the mammary gland).

As used herein “mammary epithelial cells (MECs)” refers to a luminal, basal and alveolar cells. Typical markers include cytokeratin 18, cytokeratin 14, EpCAM, progesterone receptor, estrogen receptor, prolactin receptor, Elf5 and CD24.

The mammary cell can be of a human being or any mammal but preferably a dairy animal such as cattle (cows), buffalos, goats, sheep, and camels, as well as less commonly used such as yak, water buffalo, horses, donkeys, or even reindeer. For research use also contemplated herein are mice and rats.

According to a specific embodiment, the MECs are of a bovine or human source.

The cells can be primary cells or a cell line such as an immortalized cell line e.g., genetically transformed/infected to express an immortalizing gene, e.g., SV40 or TeRT.

For example, mammary epithelial cells can be obtained from surgical explants of dissected mammary tissue (e.g., breast, udder, teat). Generally, after surgical dissection of the mammary tissue, any fatty or stromal tissue is manually removed under aseptic conditions, and the remaining tissue of the mammary gland is enzymatically digested with collagenase and/or hyaluronidase prepared in a chemically defined nutrient media, which is typically composed of ingredients that are “generally recognized as safe” (GRAS). The sample is maintained at 37° C. with gentle agitation. After digestion, a suspension of single cells or organoids is collected, either by centrifugation or by pouring the sample through a sterile nylon cell strainer. The cell suspension is then transferred to a tissue culture plate coated with appropriate extracellular matrix components (e.g., collagen, laminin, fibronectin).

Alternatively, explant specimens can be processed into small pieces, for example by mincing with a sterile scalpel. The tissue pieces are plated onto a suitable surface such as a gelatin sponge or a plastic tissue culture plate coated with appropriate extracellular matrix.

The plated cells are maintained at 37° C. in a humidified incubator with an atmosphere of 5% CO₂. During incubation, the media is exchanged about every 1 to 3 days and the cells are sub-cultured until a sufficient viable cell number is achieved for subsequent processing, which may include preparation for storage in liquid nitrogen; development of immortalized cell lines through the stable transfection of genes such as SV40, TERT (as mentioned above), or other genes associated with senescence; isolation of mammary epithelial, myoepithelial and stem/progenitor cell types by, for example, fluorescence-activated cell sorting

For example, the present inventors used a primary culture of bovine mammary epithelial cells isolated from lactating Holstein cows according to a protocol established in our lab (Cohen et al., 2015, supra).

Following are some exemplary non-limiting MECs which can be used according to the present teachings.

Immortalized human breast epithelial cells-SV40 is an immortalized breast epithelial cell line, which is not tumorigenic and does not show anchorage-independent growth. Cells in the G1, S, and G2/M phases are normally distributed, and they express normal breast epithelial markers (E-cadherin, CK7/18 and CK5/14).

Bovine mammary epithelial cell cultures are described in Jedrzejczak In Vitro Cell Dev Biol Anim. 2014; 50(5): 389-398.

HC11 mammary epithelium cell line is available from the ATCC.

Anand et al. describes the establishment and characterization of a Buffalo (Bubalus bubalis) mammary epithelial cell line wwdoi(dot)org/10(dot)1371/journal(dot)pone(dot)004046 9.

According to some embodiments of the invention, the MECs comprise ex vivo differentiated MECs. Differentiation to MECs can be from a stem cell, e.g., pluripotent stem cells or induced pluripotent stem (iPS) cells.

Hassiotou et al. Stem Cells. 2012 October; 30(10):2164-74. doi: 10.1002/stem.1188. show that human breastmilk contains stem cells (hBSCs) with mulfflineage properties. Breastmilk cells from different donors displayed variable expression of pluripotency genes normally found in human embryonic stem cells (hESCs). These genes included the transcription factors (TFs) OCT4, SOX2, NANOG, known to constitute the core self-renewal circuitry of hESCs. The methodology described in this publication represents a protocol to generate human mammary like cells and/or organoids from hBSCs.

WO2021219634 and WO2021219635 each describe methods of differentiation and culturing mammary epithelial cells in suspension. For example by i) culturing hBSCs in an appropriate culture medium (for example MammoCult medium, optionally supplemented with antibiotic-antimicotic solution and fungizone) and after one week collecting mammospheres formed thereof; and ii) growing such mammospheres in an appropriate system (such as a mammary differentiation medium comprising for example culture medium RPMI (Roswell Park Memorial Institute) 1640 with L-glutamine optionally supplemented with fetal bovine serum (FBS), insulin, epidermal growth factors (EGF), hydrocortisone, antibiotic-antimicotic solution and fungizone) for at least 1 week, for example 2 to 4 weeks, to generate lactocytes. In one embodiment of these publications, generating lactocytes comprises: i) aggregating and culturing hBSCs in an appropriate culture medium (for example MammoCult medium) in non-adherent conditions for mammospheres formation; and ii) growing such mammospheres in a 3D appropriate system (for example a mixed floating gel composed of matrix protein such as Matrigel and/or Collagen or in suspension cultures in non-adherent plates) for at least 10 days to generate lactocytes. In one embodiment, mammary commitment is obtained by applying a conditioned medium (for example EpiCultB) supplemented with specific factors (for example Parathyroid hormone (pTfirP), hydrocortisone, insulin, FGF10, and HGF). Generation of mammary—like organoids can be done by culturing the cells under conditions selected from the group consisting of: 21) monolayers of cells, 2D with attached EBs, in suspension in non-adherent plates and in mixed floating gel. Additional protocols for the generation of monolayers and organoids are provided in these publications and are incorporated by reference.

According to some embodiments, the MECs comprise a MEC monolayer. A cell monolayer is typically grown under adherent conditions, using a substrate adherent matrix or a feeder layer (stroma). However, suspension cultures are also contemplated herein (e.g., mammospheres).

It may be considered beneficial to grow the cells as a monolayer to achieve a polarity which enables nutrition of the cells from one position and collection of the secretome from another, i.e., a polar system, see e.g., WO2021142241, further described hereinbelow.

The cells can be native or transgenic cells (see Kuan et al. infra) such as modified to express a recombinant protein, e.g., antibodies.

Regardless of the method used, once MECs are obtained they are admixed with (contacted or incubated with) a medium composition comprising the tissue culture, a biologically effective concentration of a composition selectably operable to increase the secretion of the predetermined components, said composition comprising at least one agent selected from the group consisting of oleic acid, β-hydroxybutirate (BHBA) and a phenolic composition.

As used herein “medium” refers to an artificial or synthetic medium with a defined chemical structure.

Commonly used commercial growth media usually include amino acids, essential fatty acids and glucose or pyruvate, which are intended to provide the cellular nutritional needs for production of milk components.

The medium can be supplemented with lactogenic factors for example prolactin, hydrocortisone, and insulin.

For example, the medium can be a basal medium (e.g., DMEM/F12) or complex medium (e.g., RPMI-1640, IMDM) supplemented with bovine serum albumin (BSA e.g., 0.15% (v/v)) and insulin (e.g., 1 μg/ml), hydrocortisone (e.g., 0.5 μg/ml) and prolactin (e.g., 1 μg/ml) for a sufficient time, e.g., 48 h, to induce lactogenic response. Exemplary ranges are provided infra: BSA (0.001-5%); Insulin (0.001-1 μg/ml); hydrocortisone (0.05-5 μg/ml); prolactin (0.01-10 μg/ml).

As mentioned, the nutritional medium is admixed (supplemented with) a biologically effective concentration of a composition selectably operable to increase the secretion of the predetermined components, said composition comprising at least one agent selected from the group consisting of oleic acid, β-hydroxybutirate (BHBA) and a phenolic composition.

In the context of the disclosure, the term “biologically effective amount” or “biologically effective concentration” means the amount or (weigh, volume v/v or w/v or w/w) concentration of the active agent or composition needed to affect the desired biological, often beneficial, result. The amount of agent employed in the medium will be that amount necessary to deliver a biologically effective amount of the agent to achieve the desired biologic result.

As used herein “at least one” refers to 1, 2, 3, 4, 5 or more, but not more than 10 agents.

According to some embodiments of the invention, the agent promotes accumulation and/or secretion of a milk component such as described above to the collected medium, collectively referred to as a “lactogenic response”.

As used herein “oleic acid” refers to the fatty acid having the lipid number 18:1 cis-9 lipid. Oleic acid is commercially available from Merck and Avanti. Food grade oleic acid is available from Univar solutions.

The present inventors have shown that culturing MECs in oleic acid increased fat content in medium collected from cells exposed to oleic acid compared with control.

The effect of oleic acid on accumulation of fat can be determined as described hereinabove or specifically, using Nile red staining (red).

According to a specific embodiment, the effective concentration of oleic acid is about, 50-1000 μM, 100-1000 μM, 100-500 μM, 50-500 μM or 50-360 μM.

Typical culturing period can be in the range of 20 min to 36 h, e.g., 12-24 hours.

To reduce the toxic effects of oleic acid, it is preferably provided in the presence of BSA.n BSA protects against the deleterious effects of high level oleic acid e.g., above 100 uM, such as cytotoxicity, oxidative stress, apoptosis and necrosis.

Thus, according to some embodiments of the invention, a concentration of the BSA is between about 0.5-32 mg BSA/ml medium when provided with oleic acid.

According to other embodiments, the biologically effective concentration of the at least one of: oleic acid is between about 50 μM and about 360 μM, and the BSA concentration is between about 0.5 mg BSA/ml Medium and about 32 mg/ml.

As used herein “β-hydroxybutirate (BHBA)” is an organic compound and a beta hydroxy acid with the chemical formula CH₃CHCH₂CO₂H or the conjugated form.

It is commercially available from Merck, Sigma, regular or food grade.

As indicated, the lipogenic precursor BHBA is added in certain exemplary implementations, to enhance production of de novo synthesized fatty acids.

As shown in the Examples section which follows, cells were incubated with oleic acid with or without beta hydroxybutyrate. The results show that oleic acid increases triglyceride content in the cells, while addition of BHBA increases the content even further. According to a specific embodiment, the increase is synergistic.

According to a specific embodiment, the medium comprises between about 1 μM BHBA/ml medium and about 2 μM/ml, or between about 0.5 mM and about 2.5 mM, e.g., 1.2 mM. Such a concentration will enable increased production of milk fat (lipid) by the MECs.

According to a specific embodiment, the predetermined components are milk lipids.

According to a specific embodiment, the milk lipids comprise any of triglycerides, polar lipids, glycerophospholipids, sphingolipids, ceramide, gangliosides, cholesterol, lysophsphatidylcholine, lysophospholipids and free fatty acids.

According to a specific embodiment, the milk component is a milk lipid (e.g., triglycerides) and the agent is oleic acid and BHBA and optionally or alternatively a phenolic compound such as gallic acid (which increases lipid droplet accumulation in the cells) or myricetin (which increases triglyceride secretion) or derivatives thereof, as described hereinbelow.

The production of milk components requires high metabolic rates and oxygen consumption to provide energy required to support the productive and secretory state of the cells. This situation can lead to oxidative stress which results in utilization of nutrients like glucose and amino acids to produce reducing agents and reduce ATP production by the mitochondria to maintain oxidative status of the cell. The present inventor now shows that phenolic supplement allows MEC to “spare” (avoid metabolizing) glucose for lactose synthesis.

Without being bound by theory, it is suggested that the sparing effect will allow the cells to produce more lactose and eventually oligosaccharides [either human milk oligosaccharide (HMO) or bovine milk oligosaccharide (BMO), dependent on the MECs used], which utilize glucose and lactose as building blocks.

In the studies detailed below, the present inventor used a plant extract which consists of the following phenolic compounds-myrecitin, gallic acid and rutin to increase fat and protein production by mammary epithelial cells. Together this combination increased the intracellular content of milk fat, as well as secretion of fat and proteins (including casein and whey). The present inventors also determined the effect of the isolated phenolic compounds, myrecitin and gallic acid and found that each of them resulted in enhancing a specific production traits of MEC (i.e. protein, fat or lactose).

Thus, according to a specific embodiment, the agent is a phenolic composition.

As used herein “a phenolic composition” refers to a non-toxic (to consumption by human or domesticated animal) chemical which comprises at least one phenol group.

According to a specific embodiment, the phenolic composition is a derivate of gallic acid, or a glycosylated form of gallic acid or a flavanol, flavone, flavonoid, anthocyanin, or compounds known as hydrolysable or condensed tannin. Such compounds include polymers of flavonoids of high molecular weight and polyesters of gallic or ellagic acid that are bound to different sugars.

According to a specific embodiment, the composition is a polyphenol.

As used herein “polyphenols” refers to a family of organic compounds characterized by multiples of phenol groups, which are typically naturally occurring in plants.

According to a specific embodiment, the phenolic composition is selected from the group consisting of a flavonol, flavanol, flavone, flavanone and an anthocyanidin.

According to a specific embodiment, the phenolic composition is glycosylated.

Exemplary phenolic compositions which can be used in accordance with the present teachings, include, but are not limited to:

| | | | |
|---|---|---|---|
| Flavonols | (2:1) Quercetin (2:2) Kaempferol (2:3) Myricetin | R₁: H, R₂: OH, R₃: OH R₁: H, R₂: OH, R₃: H R₁: OH, R₂: OH, R₃: OH | 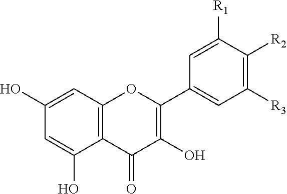 |
| Flavanol | (2:4) Catechin | R₁: H, R₂: OH, R₃: OH | 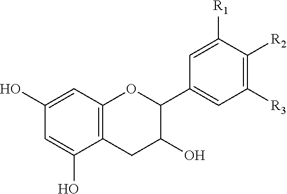 |
| Flavones | (2:5) Apigenin (2:6) Luteolin (2:7) Chrysoeriol | R₁: H, R₂: OH, R₃: H R₁: H, R₂: OH, R₃: OH R₁: OCH₃, R₂: OH, R₃: OH | 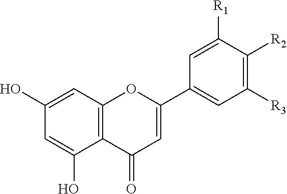 |
| Flavanones | (2:8) Hesperetin (2:9) Naringenin (2:10) Eriodictynol | R₁: H, R₂: OCH₃, R₃: OH R₁: H, R₂: OH, R₃: H R₁: H, R₂: OH, R₃: OH | 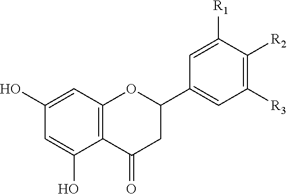 |
| Anthocyanidins | (2:11) Peonidin (2:12) Malvidin (2:13) Delphinidin (2:14) Cyanidin | R₁: OCH₃, R₂: OH, R₃: H R₁: OCH₃, R₂: OH, R₃: OCH₃ R₁: OH, R₂: OH, R₃: OH R₁: H, R₂: OH, R₃: OH | 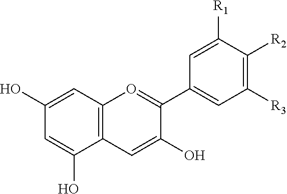 |

According to a specific embodiment, the phenolic composition is oleuropein, chlorogenic PGP-acid, catechin or myricetin such as available from Cayman Chemicals (Ann Arbor, MI, USA).

According to a specific embodiment, the phenolic composition is a galloyl derivative, favonol glucoside or catechin.

According to a specific embodiment, the phenolic composition is a dietary phenolic compound typically originating from plant derived food source, typically rich in secondary metabolites.

According to a specific embodiment, the phenolic composition is synthetic.

According to a specific embodiment, the phenolic composition is purified from a natural source, e.g., plant.

According to a specific embodiment, the phenolic composition is comprised in a plant extract, e.g., an ethanol extract, a chloroform extract, a hexane extract an ethyl acetate extract. According to a specific embodiment, the plant extract is a chloroform extract or a hexane extract. According to a specific embodiment, the plant extract is not an ethanol extract or an ethyl acetate extract of Pistacia lentiscus.

Examples of plants from which the phenolic compositions can be extracted/purified include, but are not limited to Pistacia lentiscus, Buckwheat, Japanese pagoda tree, Eucalyptus. Quebrachol.

According to a specific embodiment, the phenolic composition is gallic acid or derivative thereof, such as polyesters of gallic or ellagic acid that are bound to different sugars like epigallocatechin gallate (EGCG), catechin.

A combination of phenolic compositions can act in synergy in promoting secretion of milk components.

The present inventor has shown that the selection of the phenolic composition can affect the accumulated/secreted milk component.

For example, it is shown that polyphenolic compounds such as glycosylated polyphenols, e.g., rutin and myricetin or derivatives thereof, e.g., EGCG, catechin, can increase protein, lactose and triglyceride secretion, whilst gallic acid increases lipid droplet accumulation and lactose secretion.

It is suggested, without being bound by theory, that like pyruvate, phenolic compositions act as antioxidants, thus causing glucose sparing that eventually lead to oligosaccharide synthesis and secretion.

It is suggested that the composition of the milk components can be determined by the selected phenolic composition. Thus, for instance, glycosylated polyphenols such as myricetin can lead to protein, lactose and triglyceride secretion, while gallic acid, intracellular lipid accumulation, lipid secretion, and lactose secretion.

According to a specific embodiment, the phenolic composition is purified from a natural source, e.g., plant extract or fraction thereof (not a whole extract).

According to a specific embodiment, the phenolic composition is gallic acid or derivative thereof such as methyl-gallate, Ethyl-gallate, Catechin and Epi-Catechin.

According to a specific embodiment, the biologically effective concentration of the gallic acid is between about 1 ppm and 3 ppm.

According to a specific embodiment, the predetermined components are milk carbohydrates like lactose, 3′-galactosyllactose (Gal(β1-3)Gal(β1-4)Glc), 4′-galactosyllactose (Gal(β1-4)Gal(β1-4)Glc), 6′-galactosyllactose (Gal(β1-6)Gal(β1-4)Glc), 2′-FL (Fuc(α 1-2)Gal(β1-4)Glc), and 3-fucosyllactose (3-FL) (Gal(β1-4)[Fucα1-3]Glc), 3′-sialyllactose (3′-SL; Neu5Ac (α 2-3)Gal(β1-4)Glc) and 6′-sialyllactose (6′-SL) (Neu5Ac(α 2-6)Gal(β1-4)Glc) Gal (β1-3)GlcNAc, Lacto-N-biose, Gal (β1-4)GlcNAc, N-Acetyllactosamine. In general milk oligosaccharides consist of monosaccharides-glucose (Glc), galactose (Gal), N-Acetyl-Glucosamine (GlcNAc), fucose (Fuc), and sialic acid (Neu5Ac). These single monosaccharides are conjugated via several linkage types (i.e., glycosidic bonds).

According to a specific embodiment, the milk carbohydrate is lactose.

According to a specific embodiment, the at least one agent is gallic acid or myricetin or derivative thereof such as rutin, cathechin, ethyl gallate, EGCG.

According to a specific embodiment, the biologically effective concentration of the gallic acid is between about 1 ppm and 3 ppm.

Thus, for instance, to selectably increase the production of the milk proteins, it was found that biologically effective amount of myrecitin (e.g., between about 0.5 ppm and about 2.5 ppm, for example, between about 0.5 ppm and about 1.5 ppm, for example, 1 ppm), is operable to increase casein and whey secretion by 30% by MECs' cultures compared with control.

According to a specific embodiment, the predetermined components are milk proteins. The main milk proteins include but are not limited to alpha, beta and kappa casein, alpha lactalbumin, betalactoglobulin, lactoferin, lactadherin and lysozyme.

According to a specific embodiment, the milk protein is at least one of whey protein, α-s1, α-s2, β, and 6 casein.

According to a specific embodiment, the at least one phenolic composition is myricetin or derivative thereof. These include, but are not limited to rutin, cathechin, ethyl gallate, EGCG at 0.01 to 10 micromolar.

To improve synthesis and secretion of milk proteins the medium can be supplemented with amino acids and/or pyruvate, the latter may augment the glucose sparing effect discussed above. Pyruvate concentration range may be 0.01-4 mmol. Amino acids: lysine at a concentration range of 0.01-10 mM, methionine at 0.01-5 mM.

In certain exemplary implementations, the compositions disclosed, used in the methods described, are operable to increase production of milk proteins. Typically, and as indicated, commercial growth medium usually include amino acids, essential fatty acids and glucose or pyruvate, which are intended to provide the cellular nutritional needs for production of milk components. For example, methionine (Met) and lysine (Lys) have been identified frequently as the two most limiting AA for milk yield and milk protein synthesis. The milk proteins being at least one of: whey protein, α-s1, α-s2, β, and 6 casein.

In an exemplary implementation, myrecitin, a member of the flavonoid class of polyphenolic compounds with antioxidant activity, in a combination with methionine and lysine amino acids, at different concentrations, which are known limiting amino acids in milk protein synthesis are added. Likewise, the ratio Met/Lys is maintained at about 1:3. The AA composition used in the growth medium can be, for example, comprises Lysine (Lys), Methionine (Met), Threonine (Thr), Phenylalanine (Phe), Leucine (Leu), Isoleucine (Ile), Valine (Val), and Histidine (His) at predetermined ratios. These AA are maintained at a predetermined ratio configured to enhance production of various components. For example, the predetermined AA ratio is: Lys:Met 2.9:1; Thr:Phe 1.05:1; Lys:Thr 1.8:1; Lys:His 2.38:1; Lys:Val 1.23:1.

Moreover, it is noted, that replacement of free Met with Met-Met dipeptide, promotes asi-CN synthesis in cultured bovine MECs, and mediated by enhanced intracellular substrate availability and by activating of various gene pathways. Accordingly, and in another exemplary implementation, the amino acid is a dipeptide amino acid, for instance instead of Met a Met Met di-peptide is used such as between about 5% (w/w) and about 30% of the free methionine is replaced with the Met-Met dipeptide.

According to a specific embodiment, the agent further comprises at least one of pyruvate, amino acid and an amino acid dipeptide.

According to a specific embodiment, the amino acid (AA) agent comprises Lysine (Lys), Methionine (Met), Threonine (Thr), Phenylalanine (Phe), Leucine (Leu), Isoleucine (Ile), Valine (Val), or Histidine (His) at predetermined ratios.

According to a specific embodiment, the effective concentration of the at least one of the amino acids is each between about 45 μg AA/ml medium, and about 215 μg/ml.

According to a specific embodiment, the dipeptide is a Met-Met dipeptide.

According to a specific embodiment, the predetermined AA ratio is: Lys:Met 2.9:1; Thr:Phe 1.05:1; Lys:Thr 1.8:1; Lys:His 2.38:1; Lys:Val 1.23:1.

Nan et al. Physiol Genomics 46: 268-275, 2014. Provides general guidelines for amino acid composition and ratio which supports milk protein synthesis.

The present teachings can be implemented using compositions which allow for augmenting production of milk components.

Thus according to an aspect of the invention there is provided a composition for selectably increasing milk secretion of predetermined milk component from a tissue culture comprising mammary epithelial cells (MECs), the composition comprising a base medium and a biologically effective concentration of a composition selectably operable to increase the secretion of the predetermined component, said composition comprising at least one agent selected from the group consisting of oleic acid, β-hydroxybutirate (BHBA) and a phenolic composition, wherein said agent is not an ethanol extract of P. lentiscus; and when said agent comprises oleic acid it comprises at least two of said agents.

Such a Medium May Comprise Insulin

Insulin (0.001-1 μg/ml); hydrocortisone (0.05-5 μg/ml); prolactin (0.01-10 μg/ml), growth factors like EGF, FGF and steroid hormones like estrogen and progesterone (1-100 pg/ml), transfferin (0.2-10 ng/ml), IGF-1 (0.5-100 ng/ml). These components may be added to the base medium prior to use or prepacked. According to a specific embodiment, the medium is composed of food-grade components, and optionally sterile.

Culturing the MECs in the media described herein can be done using methods which are well known in the art.

For Example:

Sharfstein et al. Biotechnology and Bioengineering, Vol. 40, Pp. 672-680 (1992) describes a basic method for culturing MECs in extended-batch and hollow-fiber reactor cultures. Batch cultures are performed on Costar polycarbonate membrane inserts, allowing basal and apical exposure to medium. Protein production, for instance, is induced in both batch and hollow-fiber cultures in hormonesupplemented medium.

Kuan et al. J. Anim. Sci. Vol. 88, E-Suppl. 2/J. Dairy Sci. Vol. 93, E-Suppl. 1/Poult. Sci. Vol. 89, E-Suppl. 1 describe culturing of mammary gland-like structures (gland ducts, lateral buds, and alveoli). Hollow fiber bioreactors are used for large-scale mammalian cell culture to produce milk componentse, e.g., recombinant proteins. The hollow fibers provide a culture system with a high surface to volume ratio. The system allows efficient exchange of nutrients and waste products across the fiber wall.

WO20211142241 describes a cell culture system designed for the collection of milk. The system is designed to support compartmentalized secretion of the product (i.e., secretome) such that the milk or secretome is not exposed to the media that provides nutrients to the cells. In the body, milk-producing epithelial cells line the interior surface of the mammary gland as a continuous monolayer. The monolayer is oriented such that the basal surface is attached to an underlying basement membrane, while milk is secreted from the apical surface and stored in the luminal compartment of the gland, or alveolus, until it is removed during milking or feeding. Tight junctions along the lateral surfaces of the cells ensure a barrier between the underlying tissues and the milk located in the alveolar compartment. Therefore, in vivo, the tissue of the mammary gland is arranged such that milk secretion is compartmentalized, with the mammary epithelial cells themselves establishing the interface and maintaining the directional absorption of nutrients and secretion of milk.

According to this embodiment, a cell culture apparatus that recapitulates the compartmentalizing capability of the mammary gland that may be used to collect milk from mammary epithelial cells grown outside of the body. Such an apparatus can include a scaffold to support the proliferation of mammary cells at the interface between two compartments, such that the epithelial monolayer provides a physical boundary between the nutrient medium and the secreted milk. In addition to providing a surface for growth, the scaffold provides spatial cues that guide the polarization of the cells and ensures the directionality of absorption and secretion.

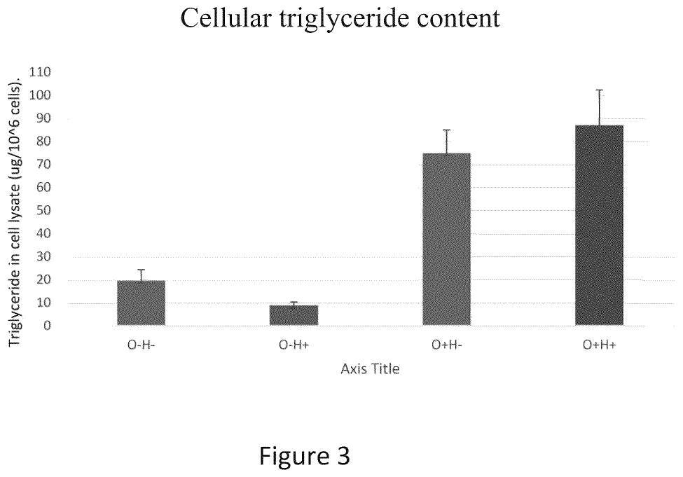

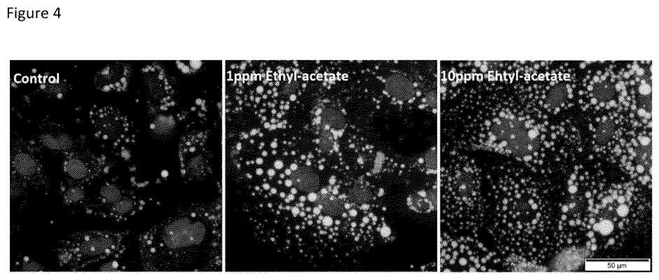

Following the isolation and expansion of mammary epithelial cells, the cells are suspended in a nutrient medium and inoculated into a culture apparatus that has been pre-coated with a mixture of extracellular matrix proteins, such as collagen, laminin, and/or fibronectin. The cell culture apparatus may be any design that allows for the compartmentalized absorption of nutrients and secretion of product from a polarized, confluent, epithelial monolayer. Examples include hollow fiber and microstructured scaffold bioreactors (sec. e.g., FIGS. 3 and 4, of WO20211142241). Alternatives include other methods of 3-dimensional tissue culture, such as the preparation of decellularized mammary gland as a scaffold, repopulated with stem cells to produce a functional organ in vitro, or collection of milk from the lumen of mammary epithelial cell organoids or “mammospheres” grown either in a hydrogel matrix or in suspension.

The apparatus includes sealed housing that maintains a temperature of about 37° C. in a humidified atmosphere of about 5% CO₂. Glucose uptake is monitored to evaluate the growth of the culture as the cells proliferate within the bioreactor. Stabilization of glucose consumption indicates that the cells have reached a confluent, contact-inhibited state. The integrity of the monolayer is ensured using transepithelial electrical resistance. Sensors monitor concentrations of dissolved O₂ and CO₂ in the media at multiple locations. A computerized pump circulates media through the bioreactor at a rate that balances the delivery of nutrients with the removal of metabolic waste such as ammonia and lactate.

Media can be recycled through the system after removal of waste using Lactate Supplementation and Adaptation technology (Freund et al. 20\IntJMol Sci. 19(2)) or by passing through a chamber of packed zeolite.

The medium can be supplemented with the agents described herein simultaneously or sequentially; or the medium can be replaced altogether.

According to a specific embodiment, cells can be cultured directly on bioreactor membranes.

Harvesting of the milk components, also referred to herein as “secretome” is done following a predetermined time in culture starting from 12 h following induction.

The secretion of milk components can be monitored as described hereinabove [e.g., staining, liquid chromatography and mass spectrophotometry (LC-MS)] and in WO20211142241.

Thus, according to an aspect of the invention, there is provided a composition comprising a secretome obtainable according to the method as described herein.

As used herein “secretome” refers to the set of proteins, carbohydrates, lipids and minerals secreted into the extracellular space by the MECs.

The present teachings also relate to secretome fractions, such as the protein fraction, lipid, carbohydrate or micronutrients such as minerals.

The composition comprising the secretome or portions thereof can be used per se, or combined with components of the food, feed or beverage industry for human or veterinary use.

Thus, according to an aspect of the invention there is provided a food or feed comprising the secretome or portions thereof.

The food can be vegan, vegetarian, dairy or may comprise meat.

As used herein “food” refers to both food (human consumption), feed (animal consumption), liquid (beverage), solid or semi-solid.

Thus, according to an aspect of the invention, there is provided a method of producing food or feed comprising combining the composition comprising the secretome or portions thereof in a food production process.

The process of producing food may include any of rising, kneading, extruding, molding, shaping, cooking, boiling, broiling, baking, frying and any combination of same.

Also provided is a method of providing nutrition to a subject in need thereof. The method comprising providing the subject with a foodstuff as described herein.

According to a specific embodiment, the subject is at risk of nutritional deficiency.

According to a specific embodiment, the subject is a healthy subject (e.g., not suffering from a disease associated with nutrition/absorption).

According to a specific embodiment the food is an “infant formula” as used herein refers to a nutritional composition intended for infants and as defined in Codex Alimentarius, (Codex STAN 72-1981) and Infant Specialities (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). The infant formulas encompass the starter infant formulas and the follow-up or follow-on formulas.

Generally, a starter formula is for infants from birth as breast-milk substitute, and a follow-up or follow-on formula from the 6th month onwards.

Other contemplated products include, but are not limited to, milk, butter, cream, cheese, ice cream, yogurt.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Material and Methods

Mammary Epithelial Cells

For all the experiments, primary culture of bovine mammary epithelial cells were used. The cells were isolated from 3 lactating Holstein cows according to an established protocol (Cohen et al., 2015 supra). Study protocols were in compliance with the regulations of the Israeli Ministry of Health, under the supervision of the Department for Control of Animal Products, State of Israel Ministry of Agriculture Rural Development Veterinary Services and Animal Health. Certificate Nu: #80. After isolation, 60,000 cells were plated in a 35-mm plastic dish on glass cover slips for all florescence staining experiments. For production analysis, 150,000 cells were plated in a 60-mm plastic dish for lipids, protein, and lactose extraction. Oxygen consumption assay was performed by using Seahorse system (Agilent, USA); cells were plated in 24-well plate (20,000 cells/well) and incubated with DMEM/F12, with 0.15% (v/v) bovine serum albumin and insulin (1 μg/ml), hydrocortisone (0.5 μg/ml) and prolactin (1 μg/ml) for 48 h to induce lactogenic response. Then cells were treated with three different P. lentiscus extracts: ethyl acetate, hexane and chloroform, according to the protocol that was used before (Azaizeh et al., 2013). The extracts were diluted with DMEM/F12 medium supplemented with Dimethyl Sulfoxide (DMSO) in a final concentration of 0.1% for better dissolution of the extractions. Oleic acid was added to the treatment medium at a final concentration of 0.1 M to increase fat (triglycerol) synthesis to better enable the visualization of intracellular lipid droplets. The treatment was given in the presence of insulin (1 μg/ml), hydrocortisone (0.5 μg/ml) and prolactin (1 μg/ml). After 24 h of incubation, medium was collected for triglyceride and phospholipid quantification, and cells were harvested for cell counting, lipid, lactose or protein extraction. Otherwise, cells were fixed for florescence staining or plates taken to seahorse for oxygen consumption rate measurement.

Intracellular Lipid Droplets Florescence Staining

After treatment, cells grown on glass were rinsed three times with phosphate buffered saline (PBS) and fixed with 4% paraformaldehyde in PBS for 20 min at room temperature. After fixation, the cover slips were rinsed three times with PBS and stained with Nile red (200 nM, sigma) for 15 min. Then cover slips were rinsed three times with PBS and stained with DAPI (Sigma, St. Louis, MO) for 5 min. Cover slips were then rinsed four times with PBS and mounted with fluorescence mounting medium (Dako, North America Inc., Carpinteria, CA). Slides were visualized with an Olympus BX40 fluorescence microscope equipped with an Olympus DP73 digital camera using CellSens Entry software (version 1.7, Olympus). Lipid droplet diameter was measured using ImageJ software (version 1.48, NIH, Bethesda, MD). The droplets were measured only after they stood in parameter of 0.1-1 circularity, and the aspect ratio was smaller than 1.5 to avoid artifacts. Differences in lipid droplets diameter between treatment are expressed in fold of change compared with control. Positive values indicate increased diameter whereas negative values indicate decreased diameter.

Milk Lipid Extraction and Analysis

After treatment, total lipids were extracted from harvested cells and from collected medium. The medium or the cells were diluted by 20 times Folch reagent (Chloroform-Methanol 2:1). After overnight incubation with cold extraction at 4° C., the upper phase was removed, and the lower phase was filtered through glass wool. Samples were then evaporated under a nitrogen stream at 65° C., diluted in 100 μl chloroform:methanol (97:3, v/v) and stored at −20° C. until injection for HPLC analysis. Separation of polar and neutral lipids was performed on a silica column (Zorbax RX-SIL, 4.6×250 mm, Agilent Technologies) using HPLC (HP 1200, Agilent Technologies, Santa Clara, CA) with an evaporative light-scattering detector (1200 series ELSD, Agilent Technologies). The column was heated to 40° C., flow was 1 mL/min, and injection volume was 20 μl. The ELSD was heated to 65° C., nitrogen pressure was 3.8 bars, filter level was set on 5, and gain (sensitivity) was set to 4 for the first 14 min, then changed to 12 until 21 min, and then changed to 7 until the end of the run, to enable detection of differently abundant lipid components. The separation protocol consisted of a gradient of dichloromethane, methanol: ammonium mix (99:1, v/v), and double-distilled water. The separation process was managed by ChemStation software (Agilent Technologies), which permitted the acquisition of data from the ELSD detector. The separated lipids were identified using external standards (Sigma Aldrich). Quantification was performed against external standard curves and expressed as μg/per 10⁶ live cells or as weight % out of the sum of phospholipids (μg) in the sample. Live cell number was determined with a hemocytometer after 5 min of trypan blue staining. Differences in triglycerides between treatment are expressed in fold of change compared with control. Positive values indicate increased triglycerides content whereas negative values indicate decreased triglycerides content.

Milk Protein Extraction and Analysis

After treatment, 0.5 ml cultured medium was collected to HPLC equipped with UV detector. Protein content was determined in 220 nm wavelength by C-18 reverse phase column. Identification and quantification were determined by constructing a calibration curve of external standard of known protein concentrations which dissolved in a 50 mM phosphate buffer (PH=6.7). Identification for α casein, β casein and α lactalbumin were qualified as caseins (α and β casein) and whey (a lactalbumin) protein. Positive values indicate increased protein content whereas negative values indicate decreased protein content.

Milk Lactose Extraction and Analysis

After treatment, 0.5 ml medium was collected to be analyzed using HPLC equipped with a refractive index detector at 68° C. Sulfuric acid (0.005N) was used for elution in 0.6 ml/min for 14 min in Rezex-ROA-acids H⁺ column. Identification and quantification were determined by establishing a calibration curve of external standard of a known lactose concentration dissolved in water. Calibration curve strength for lactose was R²=0.99. Differences in lactose between treatment are expressed in fold of change compared with control. Positive values indicate increased lactose content whereas negative values indicate decreased lactose content.

### Example 1

Oleic Acid Increases Fat Production and Secretion by Mammary Epithelial Cells

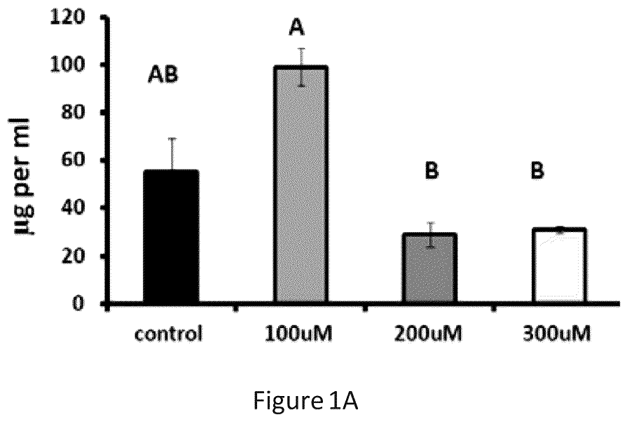

Mammary epithelial cells were incubated with 100 uM oleic acid in the presence of BSA. The triglycerides in the medium were stained with Nile red, FIGS. 1A-B clearly show higher fat content in medium collected from cells exposed to oleic acid compared with control.

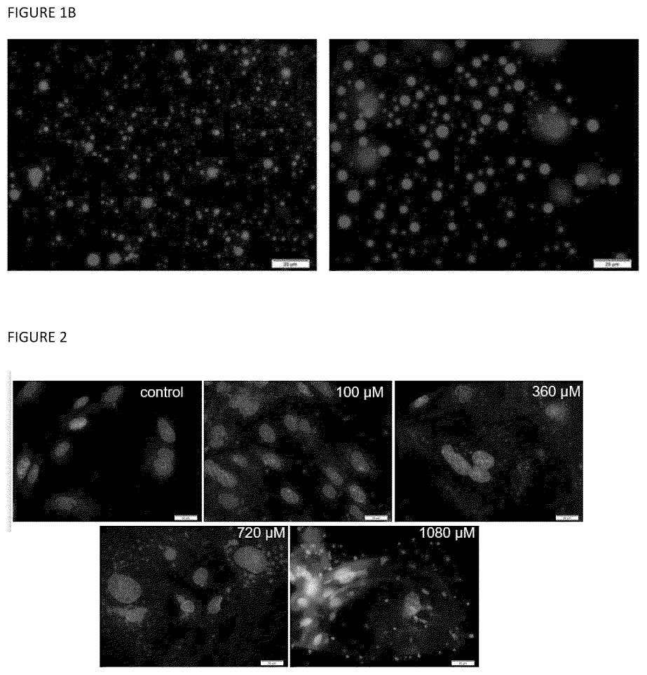

The effect of oleic acid on accumulation of fat in primary culture of mammary epithelial cells was determined using Nile red staining (red). Nucleus is stained with Dapi (blue, FIG. 2). Toxic effect is evident at oleic acid concentration of over 1 mM.

### Example 2

Combination of Beta Hydroxybutyrate and Oleic Acid Increases Fat Production by Primary Culture of Mammary Epithelia Cells

Cells were incubated with 360 uM oleic acid (O) with or without 1.2 mM beta hydroxybutyrate (BHBA, H). The concentration of triglyceride was measured in cell lysates. The results show that oleic acid increased triglyceride content in the cells by 4 fold, while addition of BHBA increased the content even further, to almost 90 ug/10⁶ cells (FIG. 3).

### Example 3

Supporting the Oxidative Status of Mammary Epithelial Cells to Enhance Production of Milk Constituents

The production of milk components requires high metabolic rates and oxygen consumption to provide energy required to support the productive and secretory state of the cells. This situation can lead to oxidative stress which results in utilization of nutrients like glucose and amino acids to produce reducing agents and reduce ATP production by the mitochondria to maintain oxidative status of the cell. The present inventor now shows that the phenolic supplement allows MEC to “spare” glucose for lactose synthesis.

It is now suggested that the sparing effect will also allow the cells to produce more lactose and eventually oligosaccharides [either human milk oligosaccharide (HMO) or bovine milk oligosaccharide (BMO)],) which utilize glucose and lactose as building blocks.

In the studies detailed below, the present inventors used a plant extract which consists of the following phenolic compounds-myrecitin, gallic acid and rutin to increase fat and protein production by mammary epithelial cells. The whole extract consisted of 3 major phenolic compounds; rutin, myrecitin and gallic acid (ethyl-acetate extract). Together this combination increased the intracellular content of milk fat, as well as secretion of fat and proteins (including casein and whey). The present inventors also determined the effect of the isolated phenolic compounds, myrecitin and gallic acid and found that each of them resulted in enhancing a specific production traits of MEC (i.e. protein, fat or lactose).

Results

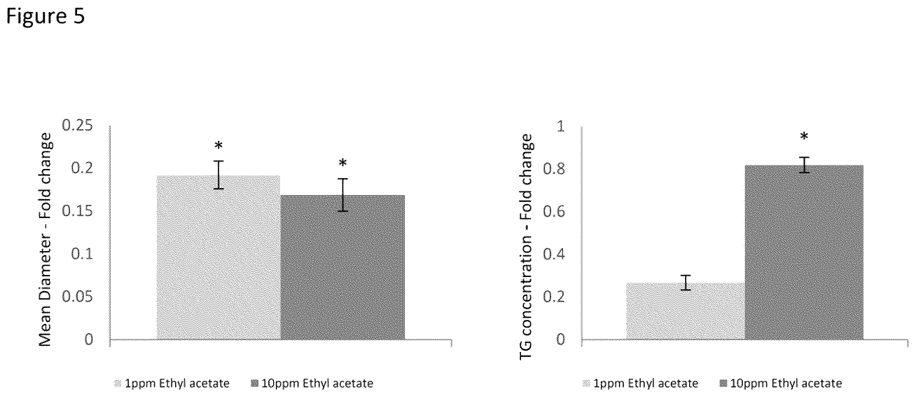

As shown in FIGS. 4 and 5, the exposure of MEC to 1 or 10 ppm of ethyl—acetate extract, which comprises rutin, gallic acid and myricetin, increased fat content in mammary epithelial cells. Also, exposure to ethyl-acetate extract resulted in increased production of milk proteins, including casein and whey, and their secretion to the media, compared with control.

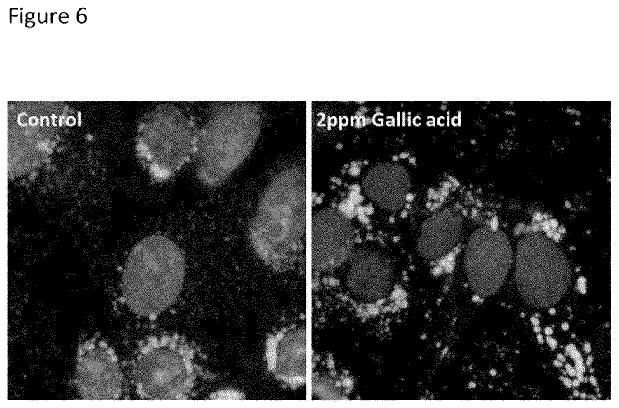

Gallic acid addition to the MEC primary culture increased the size of the intracellular lipid droplets, which is the precursor for the secreted milk fat globule (FIG. 6). Therefore, larger globules are often used as a surrogate for lipid content in the cells and for the secreted milk fat.

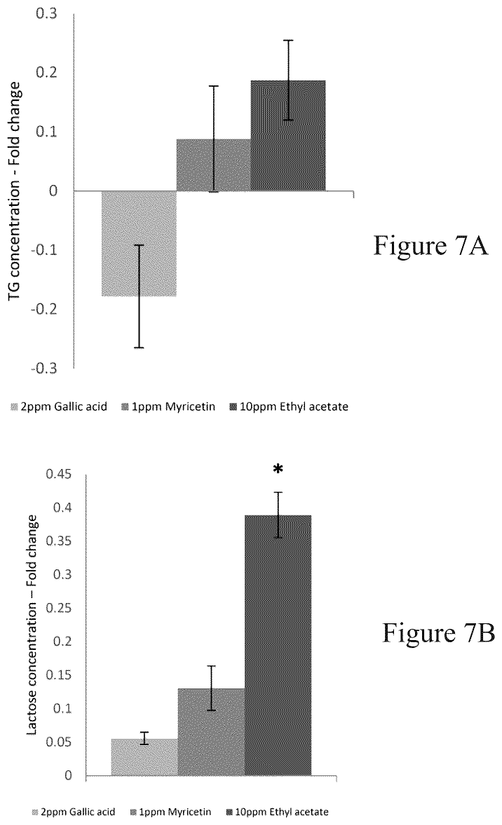

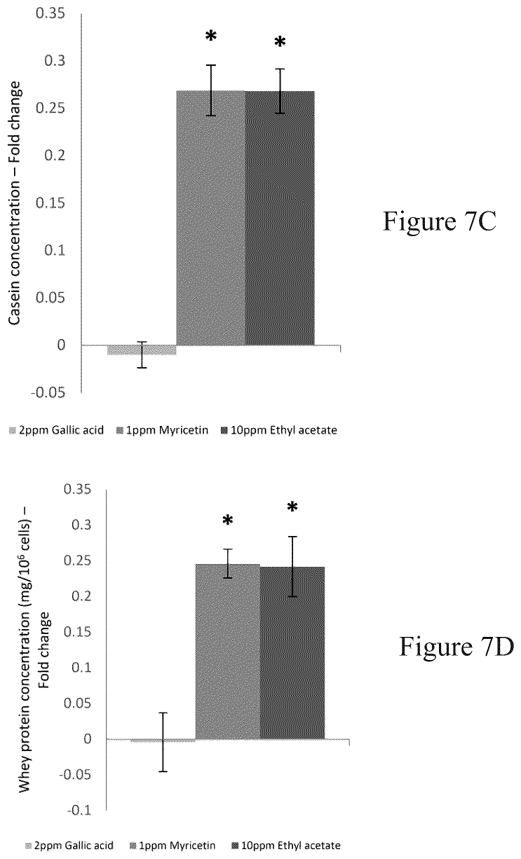

Milk proteins casein and whey secretion were 126% and 124% higher in after exposure to 1 ppm myricetin and 10 ppm ethyl acetate fraction (P<0.05, P=0.05, respectively, FIG. 7C, 7D), while exposure to 2 ppm gallic acid did not change protein secretion compared to control (P=0.98, FIG. 7C, 7D). Exposure to gallic acid and myricetin did not affect intracellular triglyceride content.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of increasing milk secretion of a predetermined milk component from a tissue culture comprising mammary epithelial cells (MECs), the method comprising admixing into a medium composition comprising the tissue culture, a biologically effective concentration of a composition selectably operable to increase the secretion of the predetermined components, said composition comprising at least one agent selected from the group consisting of oleic acid, β-hydroxybutirate (BHBA) and a phenolic composition, wherein said agent is not an ethanol extract of P. lentiscus, and when said agent comprises oleic acid it comprises at least two of said agents.

2. A composition for selectably increasing milk secretion of predetermined milk component from a tissue culture comprising mammary epithelial cells (MECs), the composition comprising a base medium and a biologically effective concentration of a composition selectably operable to increase the secretion of the predetermined component, said composition comprising at least one agent selected from the group consisting of oleic acid, β-hydroxybutirate (BHBA) and a phenolic composition, wherein said agent is not an ethanol extract of P. lentiscus; and when said agent comprises oleic acid it comprises at least two of said agents.

3. The method of claim 1, further comprising harvesting secretome of the MECs.

4. The method claim 1, wherein the predetermined components are milk lipids.

5. The method of claim 1, wherein said effective concentration of oleic acid is about 100-1000 μM or 50-360 μM.

6. The method of claim 1, wherein when said agent comprises oleic acid it comprises BSA and optionally a concentration of said BSA is between about 0.5-32 mg BSA/ml medium.

7. The method of claim 1, wherein said MECs are of a bovine or human source.

8. The method of claim 1, wherein said agent comprises at least 2 agents comprising oleic acid and BHBA and optionally the biologically effective concentration of the BHBA is between about 0.5-2.5 mM.

9. The method of claim 1, wherein said phenolic composition is selected from the group consisting of a flavonol, flavanol, flavone, flavanone and an anthocyanidin.

10. The method of claim 1, wherein said phenolic composition is gallic acid or derivative thereof and optionally the biologically effective concentration of the gallic acid is between about 1 ppm and 3 ppm.

11. The method of claim 1, wherein the predetermined components are milk carbohydrates and optionally the milk carbohydrate is lactose.

12. The method of claim 11, wherein said at least one agent is gallic acid or myricetin or derivative thereof.

13. The method of claim 1, wherein the predetermined components are milk proteins and optionally the milk protein is at least one of: whey protein, α-s1, α-s2, β, and 6 casein.

14. The method of claim 1, wherein said agent further comprises at least one of pyruvate, amino acid and an amino acid dipeptide.

15. The method of claim 14, wherein the amino acid (AA) agent comprises Lysine (Lys), Methionine (Met), Threonine (Thr), Phenylalanine (Phe), Leucine (Leu), Isoleucine (Ile), Valine (Val), or Histidine (His) at predetermined ratios.

16. The method of claim 15, wherein the effective concentration of the at least one of the amino acids is each between about 45 μg AA/ml medium, and about 215 μg/ml.

17. The method of claim 14, wherein said dipeptide is a Met-Met dipeptide.

18. The method of claim 15, wherein the predetermined AA ratio is: Lys:Met 2.9:1; Thr:Phe 1.05:1; Lys:Thr 1.8:1; Lys:His 2.38:1; Lys:Val 1.23:1.

19. A composition comprising a secretome obtainable according to the method of claim 3.

20. A method of producing food or feed comprising combining the composition of claim 19 in a food production process.
